(19) United States
(12) Patent Application Publication
(10) Pub. No.: US 2009/0068741 A1
(43) Pub. Date: Mar. 12, 2009

(54) Methods and Compositions for Improving the Health of Cells in Culture

(75) Inventors: Florence WU, Gaithersburg, MD (US); Laurel Donahue-Hjelle, Placitas, NM (US); Peggy Lio, Freehold, NJ (US); Trent Carrier, Potomac, MD (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/016,057

(22) Filed: Jan. 17, 2008

Related U.S. Application Data
(60) Provisional application No. 60/885,366, filed on Jan. 17, 2007.

(51) Int. Cl. C12N 15/00

(52) U.S. Cl. 435/440

(57) ABSTRACT

The invention relates generally to improving the growth properties of cells in culture and more specifically to accumulating beneficial mutations in the genome of cells growing in culture. Methods are disclosed for isolating cells with improved growth properties for a number of different adverse cell culture conditions which develop during prolonged culture of cells.

This application claims the benefit under 35 U.S.C. §119(e) of U.S. Provisional Application No. 60/885,366, filed Jan. 17, 2007, which is incorporated herein by reference in its entirety.

### FIELD OF THE INVENTION

The invention relates generally to improving the growth properties of cells in culture and more specifically to accumulating beneficial mutations in the genome of cells growing in culture.

### BACKGROUND INFORMATION

Cells are grown in culture for a variety of reasons including the production of proteins, peptides and hormones. In order to maximize the production of these materials, the cells are often grown at high densities for prolonged periods of time. Cell culture media provides the nutrients necessary to maintain and grow cells in a controlled, artificial and in vitro environment. Characteristics and compositions of cell culture media vary depending on the particular cellular requirements. Important parameters include osmolality, pH, and nutrient formulations. In order to maintain the bioproduction and growth of the cells, the culture media is often supplemented during the culture period. As the cells catabolize nutrients, the environment in which the cells grow is constantly being altered. Catabolic products may remain in culture or may require the cultured cells to catabolize these also to maintain cell health. The medium is thus constantly changing. The requirements of the cultured cells may be changing also. Especially for media optimized for a particular cell type or especially a particular production task, as the cells grow (and produce) the medium becomes less conducive to the desired result. Supplementation of culture medium has been effectively used to prolong culture times or to maintain or improve production. Several supplementation programs have been used. For example, a single bolus or multiple boli have been added to culture to replenish or sometimes modify medium constituents. Continuous feed programs have also been tried. Supplementation of the growing culture can maintain growth and productivity of the cultured cells over extended time periods.

Despite the efforts to optimize culture conditions for maximum production of a desired product, there remains a need to further increase the production efficiency of cells in culture. An alternative approach to improving culture conditions is to improve the characteristics of the cultured cells themselves.

### SUMMARY OF THE INVENTION

The present invention relates to methods and compositions for modifying cells so that they are better able to withstand adverse conditions which develop when cells are grown at high density for prolonged periods of time. The present invention allows the development of cell lines which are able to maintain their growth and ability to produce biomolecules even under unfavorable culture conditions (e.g., as culture conditions become unfavorable over time).

Among the properties of culture medium which change, for example, over time, is osmolality. Osmolality may change, for example, as a consequence of products produced by cells accumulating in the media and as a consequence of supplements being added to the media. When supplements are added to media, they may be added over the duration of all or a portion of (e.g., from about 1% to about 80%, from about 1% to about 60%, from about 1% to about 50%, from about 1% to about 30%, from about 1% to about 20%, from about 1% to about 10%, from about 1% to about 5%, from about 4% to about 80%, from about 4% to about 60%, from about 4% to about 50%, from about 4% to about 30%, from about 4% to about 20% from about 4% to about 10%, from about 10% to about 80%, from about 10% to about 60%, from about 10% to about 50%, from about 10% to about 30%, etc.) the culture period.

Osmolality may increase or decrease during the cell culture process. Osmolaltity changes may occur as a result of, for example, utilization of culture media materials by cells, introduction of materials by cells into culture media, the addition of materials (e.g., one or more supplements) to the culture media, and other factors. Typically, as compared to the starting osmolaltity, the osmolality of culture media used in the practice of the invention will increase or decrease at any time during the culture process not more than 5%, 10%, 20%, 30%, 40%, 50%, 60%, 70%, 80%, or 90% (e.g., from about 5% to about 90%, from about 5% to about 80%, from about 5% to about 70%, from about 5% to about 60%, from about 5% to about 50%, from about 5% to about 40%, from about 5% to about 30%, from about 5% to about 25%, from about 5% to about 20%, from about 5% to about 15%, from about 5% to about 10%, from about 10% to about 90%, from about 10% to about 80%, from about 10% to about 80%, from about 10% to about 60%, from about 10% to about 50%, from about 10% to about 40%, from about 10% to about 30%, from about 10% to about 20%, from about 20% to about 90%, from about 20% to about 70%, from about 20% to about 50%, from about 20% to about 40%, from about 20% to about 30%, from about 30% to about 90%, from about 30% to about 60%, from about 30% to about 50%, from about 30% to about 40%, etc.)

Other properties of culture media, such as pH, may also change over time. In many instances, a cell which is able to maintain favorable growth characteristics even in a hyperosmotic environment would be desirable. Normal, or isoosmotic, is defined as 290 mOsm/kg. The osmolality of a typical culture medium for mammalian cells may be from about 260 mOsm/kg to about 320 mOsm/kg.

It has been observed in many instances that when cells are exposed to hyperosmotic conditions (350-800 mOsm/kg) populations of cells emerge which are adapted to the hyperosmotic conditions. Whether this is due to the cells becoming metabolically adapted to the hyerosmotic conditions or to selection of genetically modified cells has not been clear. The present invention provides methods for isolating cells with improved growth and production properties under non-optimal culture conditions. Non-optimal culture conditions include any conditions that impact the ability of the cells to grow, replicate and produce molecules of interest. These include, but are not limited to, low or high osmolality, low or high pH, lack of essential nutrients or growth factors, accumulation of metabolic byproducts, high or low temperature, presence or lack of a solid support for cell attachment, exposure to high shear forces, low oxygen tension, high carbon dioxide concentration, etc.

The methods disclosed may be applied to cells of any origin including plant cells, animal cells, insect cells and bacteria.

One approach to isolating cells with one or more favorable genetic modification is to increase the rate of mutation. Among the mechanisms of mutation in a cell, is base pair mismatch induced by DNA-polymerases during replication. The consequences of DNA base pair mismatch may be minimized in many cells by a DNA mismatch repair (MMR) system. Disruption of the MMR system may lead to cells with an increased mutation rate and a broader range of phenotypes. Of course, other means of increasing the mutation rate may also be used. These methods include but are not limited to the use of chemical or physical mutagens such as alkylating agents or UV light. Increasing the mutation rate may be accomplished by modulation of MMR or with other methods. Any of these methods, including modulation of MMR, may be used either alone or in combination.

Methods for modulating the MMR system are described, inter alia, in U.S. Pat. Nos. 6,576,468 and 6,825,038 which are incorporated herein in their entirety by reference. Described are methods for both reducing or eliminating MMR function and methods for restoring MMR function so that isolated mutant cells are genetically stable. Cells which are naturally deficient in MMR may also be used and MMR function restored after the useful genetic variant is isolated.

In specific embodiments of the invention, cells are subjected to a process which induces mutations and are exposed to what are typically unfavorable conditions for those cells (e.g., a hyperosmotic environment or a hypoosmotic environment). A hyperosmotic environment is one where the osmolality is from about 350 to about 800 mOms/kg, from 400 to 800, from 450 to 800, from 500 to 800, from 550 to 800, from 600 to 800, from 650 to 800, from 700 to 800, from 750 to 800, from 350 to 750, from 350 to 700, from 350 to 650, 350 to 600, from 350 to 550, from 350 to 500, from 350 to 450, or from 350 to 400 mOsm/kg. In one embodiment of the invention, the MMR system of the cells is disrupted by any method known in the art and the cells exposed to a hyperosmotic environment.

The cells may be exposed to unfavorable conditions (e.g., a hyperosmotic environment) for any of a variety of periods of time. For example, cells may be exposed to unfavorable conditions for from about 10 population doublings (PDL) to about 60 PDL. In some embodiments, the cells may then be exposed for 10 to 50 PDL, from 10 to 40, from 10 to 30, from 20 to 60, from 30 to 60, or from 20 to 40 PDL. At the end of the exposure period, the cells may be assessed for their growth rate and production rate of any desired biomolecules along with any other cellular properties that may be of interest.

In specific embodiments of the invention, the mutation rate of the cells may be increased by modulation of MMR or other methods, alone or in combination, and then the cells may be exposed to an unfavorable culture condition including, but not limited to, low osmolality, low or high pH, lack of essential nutrients or growth factors, accumulation of metabolic byproducts, high or low temperature, presence or lack of a solid support for cell attachment, exposure to high shear forces, low oxygen tension, and high carbon dioxide concentration. The cells may then be exposed to the unfavorable culture condition for from 10 to 50 PDL, from 10 to 40, from 10 to 30, from 20 to 60, from 30 to 60, or from 20 to 40 PDL. At the end of the exposure period, the cells may be assessed for their growth rate and production rate of any desired biomolecules along with any other cellular properties that may be of interest.

In alternate embodiments, the mutation rate of the cells may be increased by modulation of MMR and/or mutagenesis to produce a population of genetically diverse cells which may then be exposed to the unfavorable culture condition. The length of exposure to both the high mutation rate and to the unfavorable culture conditions may be for 10 to 60 PDL, for 10 to 50 PDL, from 10 to 40, from 10 to 30, from 20 to 60, from 30 to 60, or from 20 to 40 PDL. When both of these steps have been completed, cells with the desired properties may be selected.

In some embodiments, the disclosed methods may be used to develop cells that are resistant to multiple unfavorable culture conditions. For example, cells may be selected which are resistant to both hyperosmotic media and high pH, or hyperosmotic media and low temperature etc. This may be accomplished by using the described methods in series. For example, cells which are resistant to hyperosmotic media may be selected by increasing the mutation rate in the presence of the hyperosmotic media and then those cells may be further exposed to increased mutation rate and, for example, pH greater than 7.4. This process may be repeated any number of times (e.g., two, three, four, five, etc.) so that the resulting cells may be resistant to multiple (e.g., two, three, four, five, etc.) adverse culture conditions. Thus, the invention includes cells lines with more than one characteristic which allows them to produce macromolecules and efficiently grow under what are typically considered to be unfavorable conditions relative to the original unmodified cells.

In many instances, cells generated by methods of the invention will have a doubling time which is shorter than that of their parent/unmodified counterpart. In many instances, this shorter doubling time will be exhibited under unfavorable conditions (e.g., the unfavorable condition used to obtain the modified cells). The doubling time increase of the modified cells, as compared to their parents, may be in the range of from about 1% to about 200%, from about 1% to about 150%, from about 1% to about 100%, from about 1% to about 50%, from about 1% to about 30%, from about 1% to about 20%, from about 1% to about 10%, from about 5% to about 200%, from about 10% to about 200%, from about 20% to about 200%, from about 30% to about 200%, from about 40% to about 200%, from about 50% to about 200%, from about 10% to about 200%, from about 10% to about 100%, from about 10% to about 50%, from about 20% to about 200%, from about 20% to about 100%, from about 20% to about 80%, from about 30% to about 200%, from about 30% to about 100%, from about 30% to about 60%, from about 4% to about 200%, from about 40% to about 150%, from about 40% to about 100%, from about 40% to about 800%, from about 50% to about 200%, from about 50% to about 100%, etc.

An exemplary embodiment of the invention may be a method for improving the growth properties of cells in culture comprising: a) culturing cells under conditions wherein the mutation rate is increased; b) adjusting the culture conditions such that a parameter is not optimal for the cell; c) decreasing the mutation rate of the cells; and d) isolating cells which maintain growth and viability under the adjusted culture conditions.

### BRIEF DESCRIPTION OF THE DRAWINGS

FIG. 1 shows a representative flow chart of two embodiments of the present invention. The ‘Evolve’ step refers to disruption of MMR activity and the ‘Cure’ step refers to the restoration of MMR activity. The listed PDL values are suggested starting points and may be varied as needed.

FIG. 2 shows the growth curve for DG44 cells at different osmolalities.

FIG. 3 show the viability of DG44 cells over time at different osmolalities.

FIG. 4 shows the glucose consumption of DG44 cells over time at different osmolalities.

FIG. 5 shows the lactate production of DG44 cells over time at different osmolalities.

FIG. 6 shows the growth curve of cells adapted for growth at 450 mOsm/kg and then placed in media with osmolalities of 475, 500 and 550 mOsm/kg.

FIG. 7 shows a comparison of growth of DG44 cells at four different osmolalities.

FIG. 8 shows total viable cells over time in DG44 cells grown at an osmolality of 500 mOsm/kg.

FIG. 9 shows the growth of DG44 cells adapted for growth in 450 mOsm/kg media in 300 mOsm/kg media and the effect of switching to a higher osmolality.

FIG. 10 shows the growth of DG44 cells, in which the MMR system has been inactivated, in 300 mOsm/kg media.

FIG. 11, shows the growth of DG44 cells, in which the MMR system has been inactivated, in 450 mOsm/kg media which was then increased to 500 and then 550 mOsm/kg.

FIG. 12 shows the growth of DG44 cells, in which the MMR system has been inactivated, in 450 mOsm/kg media which was then increased to 500 mOsm/kg.

FIG. 13 shows the growth of DG44 cells, in which the MMR system has been inactivated, in 550 mOsm/kg media.

FIG. 14 shows the growth of DG44 cells, in which the MMR system has been inactivated, in 600 mOsm/kg media.

FIG. 15 shows the growth of DG44 cells transfected with the pEF1-V5-HisA plasmid and with or without inactivation of the MMR system.

FIG. 16 shows the growth and IgG production of DG44 cells adapted for growth at 500 mOsm/kg and subsequently transfected with a plasmid containing an IgG gene.

### DETAILED DESCRIPTION OF THE INVENTION

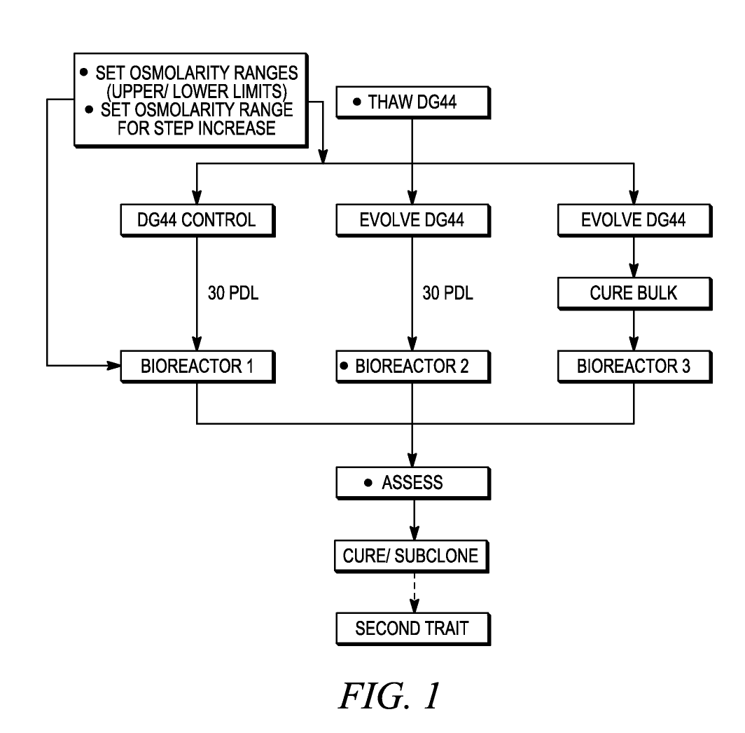

The present invention involves, in part, isolating genetically modified cells which have improved growth characteristics in culture. Two possible embodiments of the invention are outlined in FIG. 1. In one of these embodiments the cells may be exposed to a high mutation rate to develop a population of genetically diverse cells.

The high mutation rate may achieved by inactivating the MMR system in the cells using methods described in U.S. Pat. Nos. 6,576,468 and 6,825,038. Polynucleotides encoding a dominant negative form of a mismatch repair protein may be introduced into any eucaryotic cell. The gene can be any dominant negative allele encoding a protein, which is part of a mismatch repair complex, for example, PMS2, PMS1, MLH1, GTBP, MSH3 or MSH2. The dominant negative allele can be naturally occurring or made in the laboratory. An example of a dominant negative allele of a mismatch repair gene is the human gene hPMS2-134, which carries a truncation mutation at codon 134. The mutation causes the product of this gene to abnormally terminate at the position of the 134th amino acid, resulting in a shortened polypeptide containing the N-terminal 133 amino acids.

The polynucleotide can be in the form of genomic DNA, cDNA, RNA, or a chemically synthesized polynucleotide. The polynucleotide can be cloned into an expression vector containing a constitutively active promoter segment (such as but not limited to CMV, SV40, EF-1 or LTR sequences) or to inducible promoter sequences such as the tetracycline, or ecdysone/glucocorticoid inducible vectors, where the expression of the dominant negative mismatch repair gene can be regulated. When an inducible promoter is used the expression can be turned on or off by adding or removing the inducing agent. Alternatively the cells transfected with an expression vector can be cured of the plasmid using methods well known in the art. Vectors lacking a functional origin of replication may also be used so that the vector does not replicate and is lost from the culture as the cells divide. The polynucleotide can be introduced into the cell by transfection.

In addition to, or as an alternative to, inactivation of the MMR system, cells may be exposed to chemical or physical mutagenic agents such as alkylating agents, UV light or ionizing radiation. The following chemical mutagens are useful, as are others not listed here, according to the invention. N-ethyl-N-nitrosourea (ENU), N-methyl-N-nitrosourea (MNU), procarbazine hydrochloride, chlorambucil, cyclophosphamide, methyl methanesulfonate (MMS), ethyl methanesulfonate (EMS), diethyl sulfate, acrylamide monomer, triethylene melamin (TEM), melphalan, nitrogen mustard, vincristine, dimethylnitrosamine, N-methyl-N′-nitro-Nitrosoguanidine (MNNG), 7,12 dimethylbenz (a) anthracene (DMBA), ethylene oxide, hexamethylphosphoramide, bisulfan. In some embodiments of the invention, a mutagenesis technique is employed that confers a mutation rate in the range of 1 mutation out of every 100 genes; 1 mutation per 1,000 genes. The use of a combination (MMR deficiency and chemical mutagens) will allow for the generation of a wide array of genome alterations (such as but not limited to expansions or deletions of DNA segments within the context of a gene's coding region, a gene's intronic regions, or 5′ or 3′ proximal and/or distal regions, point mutations, altered repetitive sequences) that are preferentially induced by each particular agent.

Once the genetically diverse population of cells has been generated, the cells may then be grown under adverse culture conditions. These conditions include, but are not limited to, high or low osmolality, high or low pH, lack of essential nutrients or growth factors, accumulation of toxic metabolic products, high or low temperature, presence or lack of a solid support for cell attachment, high or low oxygen tension, high carbon dioxide concentration, growth in media lacking animal derived proteins etc.

What is meant by adverse conditions are those conditions that differ from those that produce normal cell doubling times for the cells being cultured. A normal cell doubling time may vary depending on the particular cell being studied but may easily be determined by one skilled in the art. For osmolality, normal, or isoosmotic, is defined as 290 mOsm/kg. The osmolality of a typical culture medium for mammalian cells may be from about 260 mOsm/kg to about 320 mOsm/kg. The pH of a typical culture medium for mammalian cells may be from about 7.2 to about 7.4. Thus high pH for a mammalian cell may be a pH of 7.5, 7.6, 7.7, 7.8, 7.9, 8.0 or greater. Low pH for a mammalian cell may be a pH of 7.1, 7.0, 6.9, 6.8, 6.7, 6.6 or less.

Mammalian cells are typically cultured at a temperature of 37° C. in the presence of 5% CO₂. Thus low temperature for a mammalian cell may be 36° C., 35° C., 34° C., 33° C., 32° C. or less. High temperature for a mammalian cell may be 38° C., 39° C., 40° C., 41° C., 42° C., or greater. A high carbon dioxide concentration for a mammalian cell may be 6%, 7%, 8%, 9%, 10% or greater.

Essential nutrients or growth factors which may become depleted during the culture of mammalian cells include but are not limited to one or more amino acids, biotin, folic acid, nicotinamide, p-amino benzoic acid, pantothenic acid, pyridoxine HCl, riboflavin, vitamin B-12, glucose, sodium pyruvate, insulin, and selenium.

Toxic metabolic by-products which may accumulate during culture of mammalian cells include but are not limited to ammonia and lactate.

In some embodiments, the high mutation rate is reduced before the cells are shifted to the adverse culture conditions. The mutation rate is reduced by removal or cessation of any mutagenic treatments and restoring the normal function of the MMR system.

Aspects of the invention are illustrated by the following examples.

The following examples are intended to illustrate but not limit the invention.

### EXAMPLES

The following examples used the CHO cell line DG44. Standard culture conditions were CD-DG44 Media (Invitrogen Corp., Carlsbad, Calif.) supplemented with 8 mM L-glutamine and 0.18% Pluronic F-68. The cultures were maintained in shaker flasks under 5% carbon dioxide and 80% relative humidity at 37° C. The osmolality of the media was approximately 300 mOsm/kg. For experiments needing a higher osmolality, sodium chloride was added as needed to achieve the desired osmolality.

### Example 1

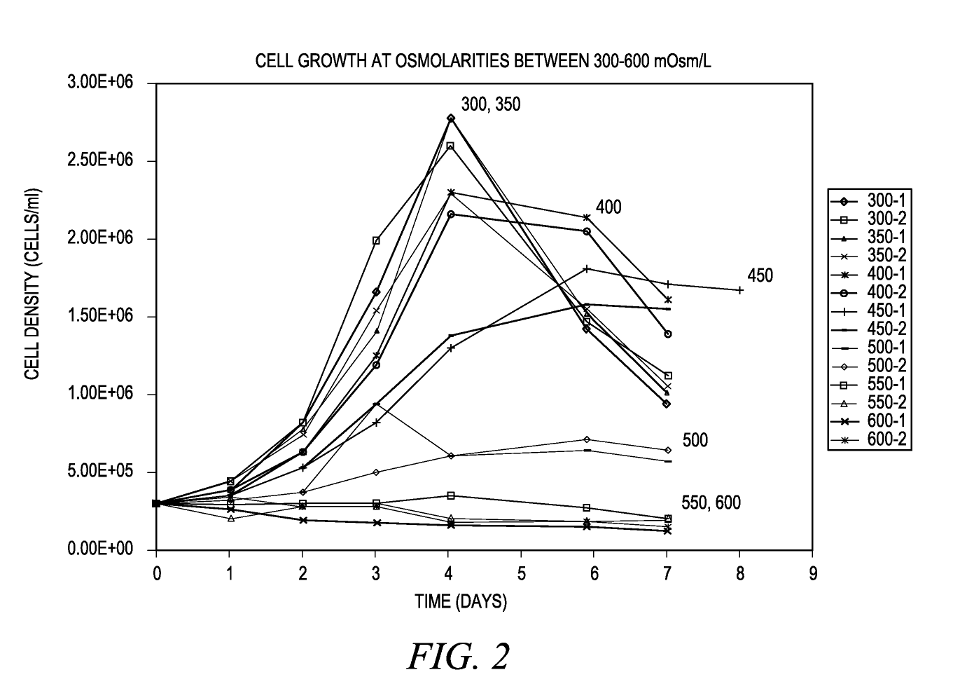

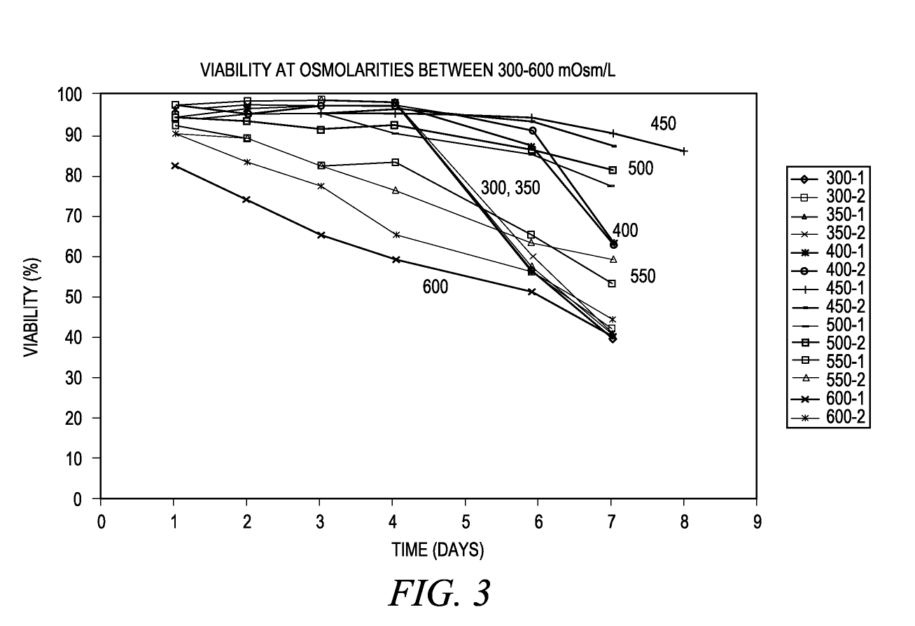

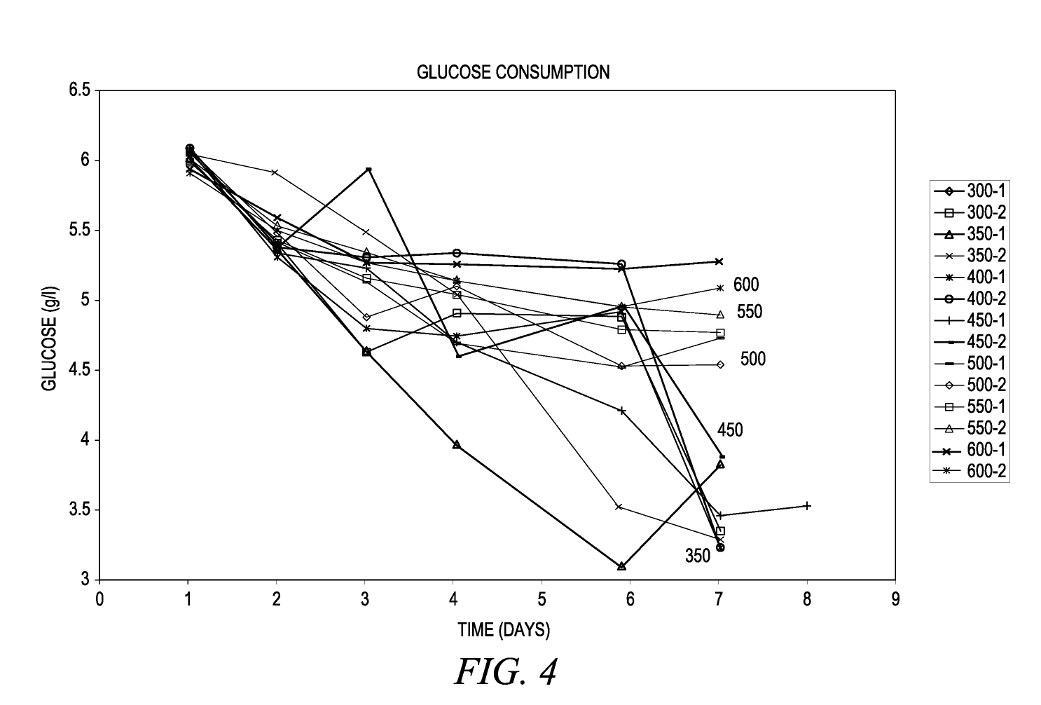

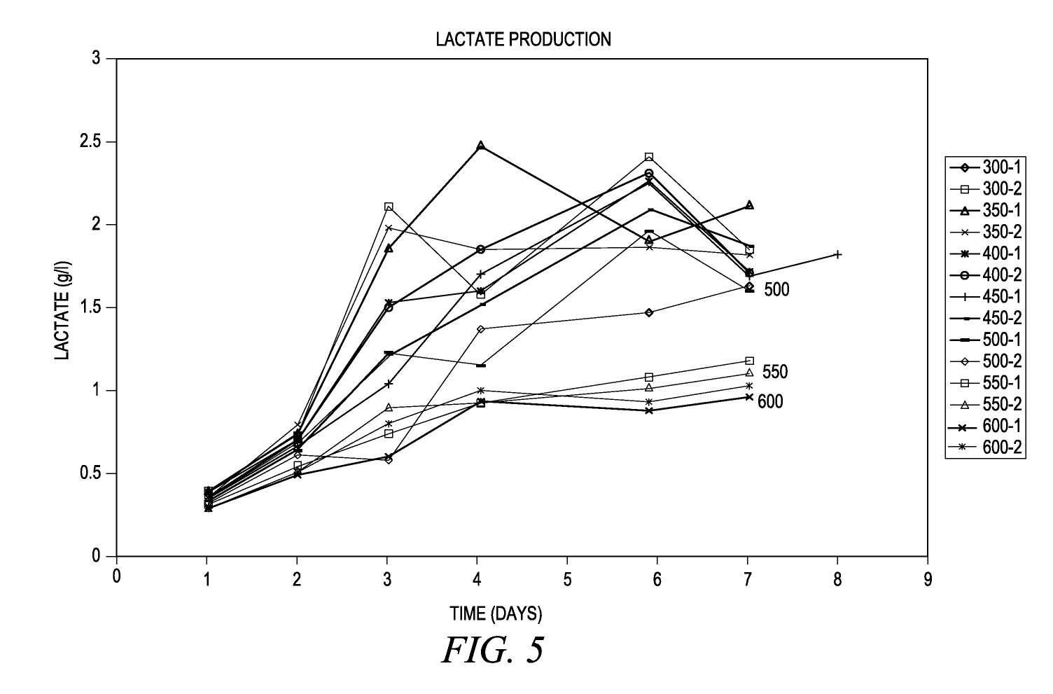

In the first experiment, DG44 cells were grown at an osmolality of from 300 to 600 mOsm/kg and several growth parameters measured. In FIGS. 2-5, total cells, cell viability, gluconse concentration and lactate levels are plotted as a function of time. Cells grown at lower osmolalities showed the most rapid growth through day four when nutrients became depleted (glucose levels, FIG. 4) and cells began to die. Cells grown at the highest osmolalities failed to show any growth over the seven days of the experiment.

### Example 2

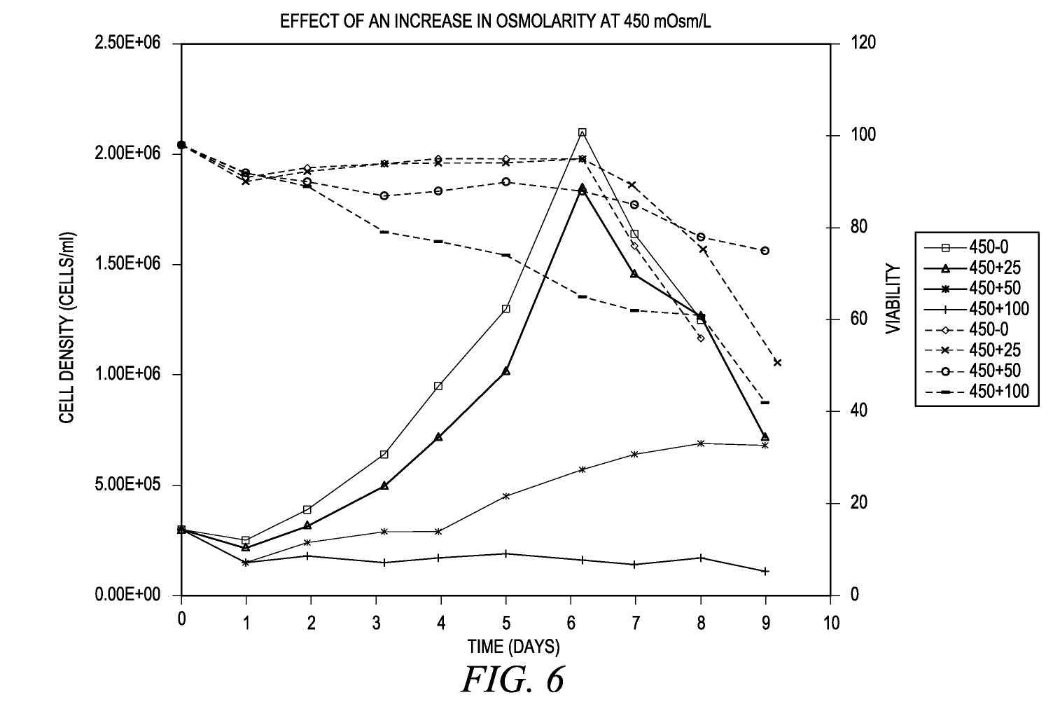

DG44 cells which had been adapted for growth at 450 mOsm/kg by disruption of the MMR system as described above were cultured at 475, 500 and 550 mOsm/g and the cell density and viability monitored. The cell density and viability of these cells was monitored for nine days and is shown in FIG. 6. DG44 cells which were kept at the adapted osmolality of 450 mOsm/kg showed good growth and viability for the first 6 days. An increase of 25 mOsm/kg only slightly reduced growth and viability. Cells subjected to an increase of 50 mOsm/kg had slow growth and good viability but those subjected to an increase of 100 mOsm/kg failed to show any growth and had reduced viability.

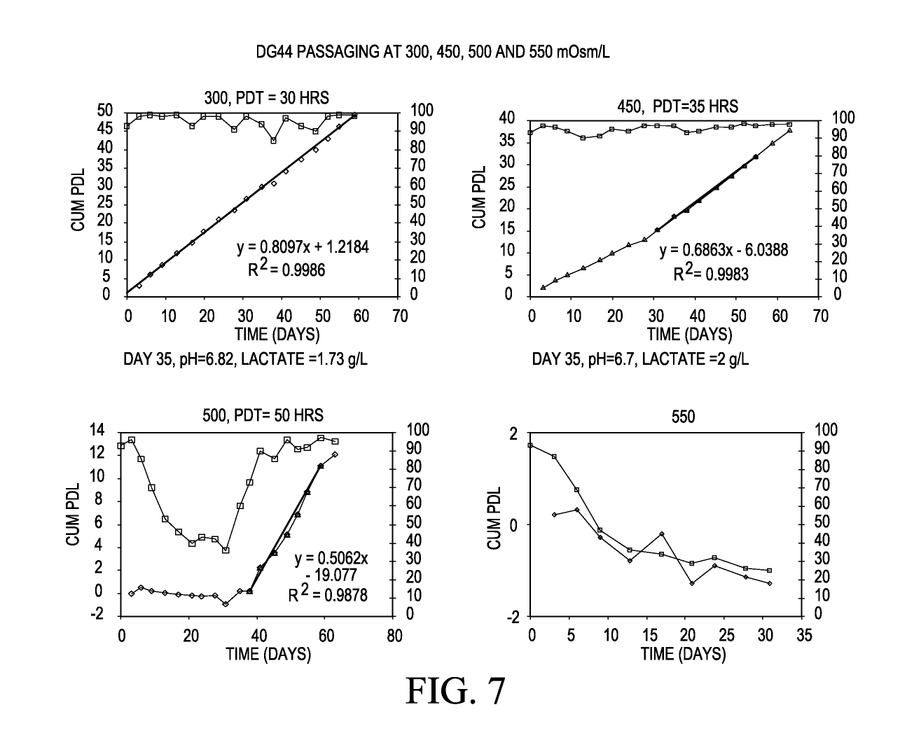

Growth and viability of DG44 cells grown at an osmolality of 300, 450, 500 or 550 mOsm/kg is shown in FIG. 7. The slope of the plot of cumulative PDL vs. time is a measure of growth rate, the higher the slope the higher the growth rate. When grown at 300 mOsm/kg, the slope was 0.8097 which decreased to 0.5062 for cells grown at 500 mOsm/kg. Cells cultured at 550 mOsm/kg failed to grow at all.

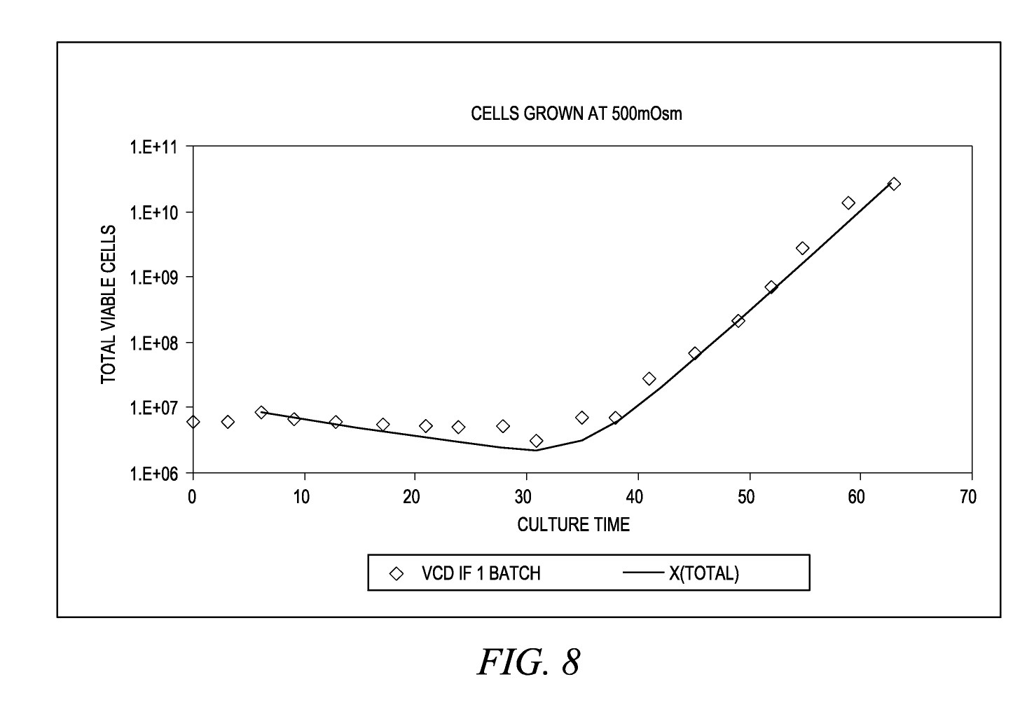

The data for 500 mOsm/kg in FIG. 6 was further analyzed by plotting the total viable cells over time (FIG. 8). Total viable cells were calculated using the following formula:

Xₜₒₜₐₗ=X_{isoosmotic}+X_{hyperosmotic}

Where Xᵢ=X₀exp^{(μ-ν)t}

and μ=growth rate, ν=death rate and t=time.

Looking at the data in FIG. 8, the cells remained stagnant for the first thirty days after which the subpopulation of cells which were tolerant of the 500 mOsm/kg osmolality became established and began to outgrow the non-adapted cells.

### Example 4

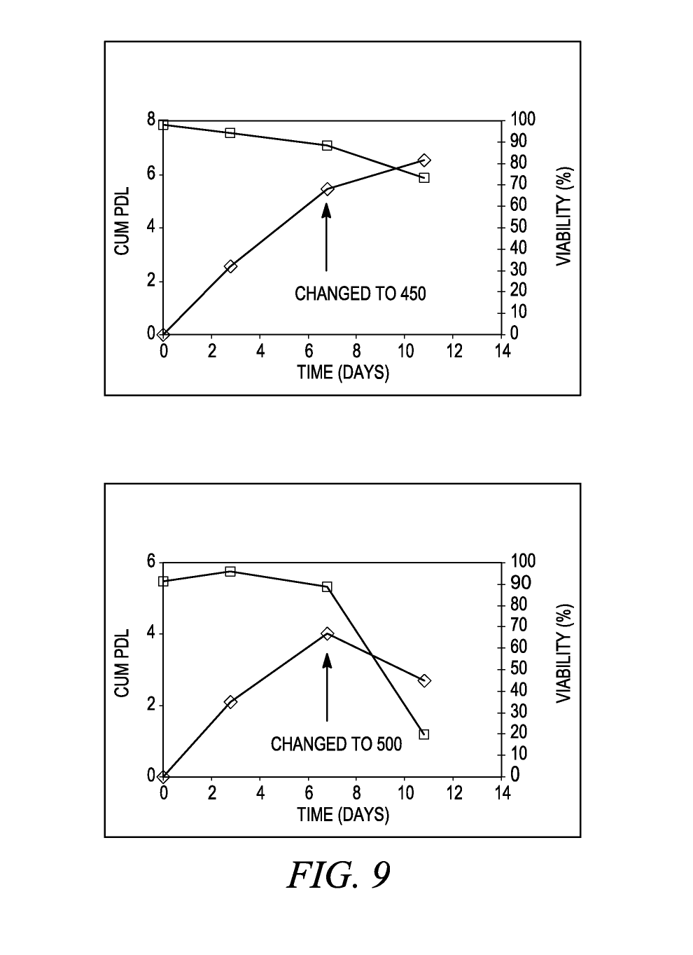

To determine if DG44 cells which had become adapted for growth at high osmolality could grow at physiologic osmolality, cells adapted for growth at 450 mOsm/kg were placed in 300 mOsm/kg media and then the media shifted back to 450 or 500 mOsm/kg after 6 days. The data from this experiment are shown in FIG. 9. The high osmolality adapted cells grew well and maintained viability for 6 days. Shifting the cells back to 450 mOsm/kg had only a modest effect on growth and viability but shifting the cells to 500 mOsm/kg led to a marked reduction in viability and cell growth.

### Example 5

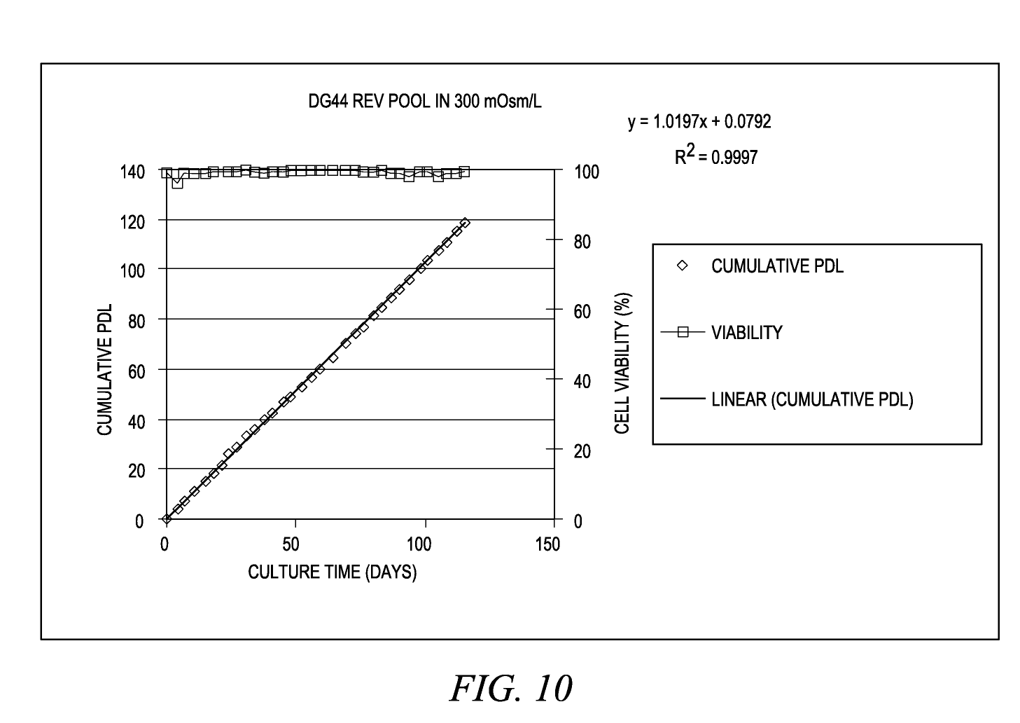

DG44 cells in which the MMR system had been disrupted as described in U.S. Pat. Nos. 6,825,038 and 6,576,468 were cultured in 300 mOsm/kg media for 115 days. The growth and viability of the culture are shown in FIG. 10. Disruption of the MMR system does not appear to adversely affect the growth of DG44 cells.

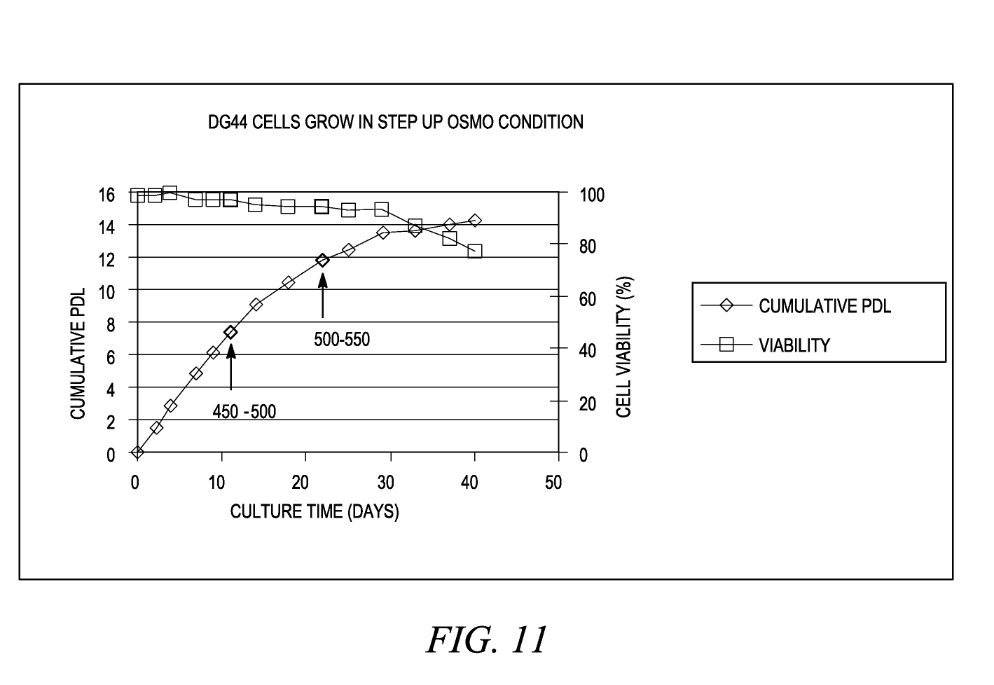

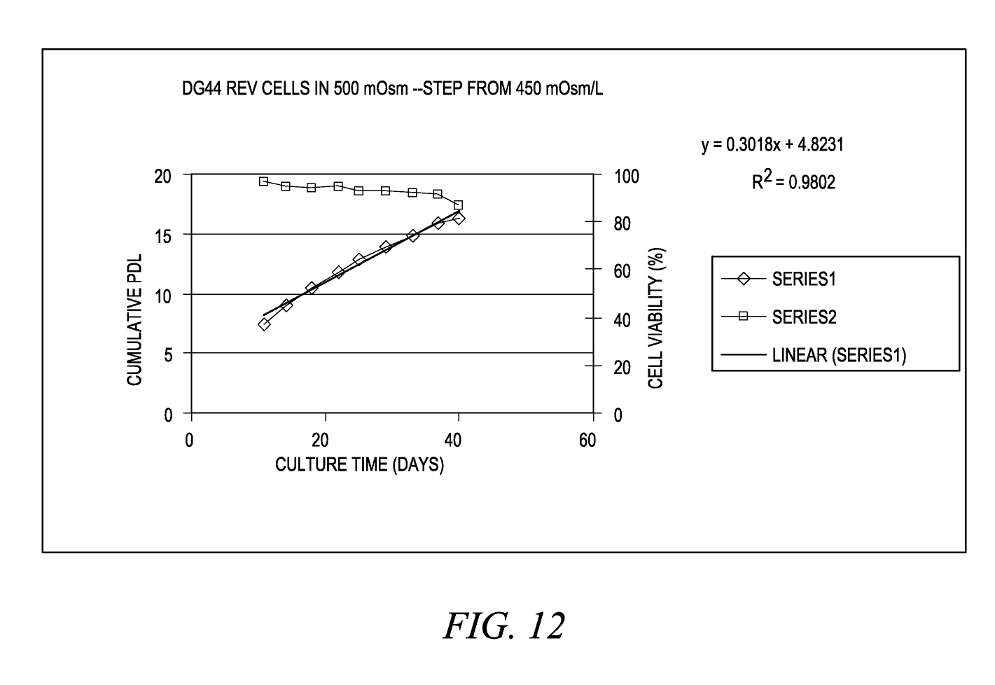

DG44 cells in which the MMR system had been disrupted as described in U.S. Pat. Nos. 6,825,038 and 6,576,468 were cultured in 450 mOsm/kg media for 11 days followed by a step up to 500 mOsm/kg for an additional 11 days when the osmolality was stepped up to 550 mOsm/kg. The growth and viability of this culture are shown in FIG. 11. A portion of the culture was maintained at 500 mOsm/kg through day 40, the growth and viability of this culture is shown in FIG. 12. Even with the MMR system disrupted, DG44 cells are able to maintain viability and growth in a hyperosmotic environment.

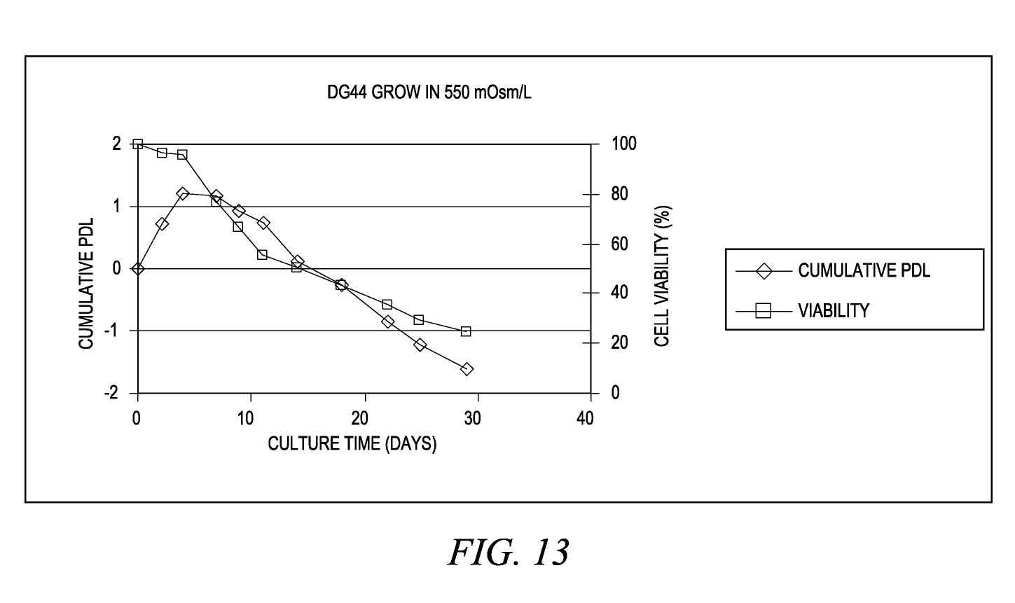

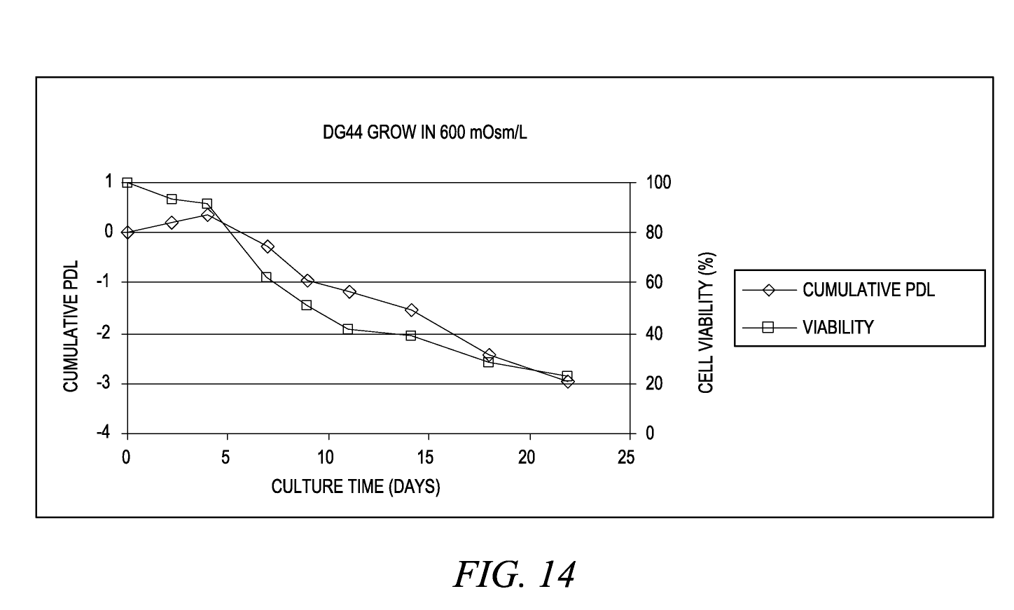

DG44 cells in which the MMR system had been disrupted as described in U.S. Pat. Nos. 6,825,038 and 6,576,468 were cultured in 550 or 600 mOsm/kg media. The growth and viability of these cultures is shown in FIGS. 13 and 14 respectively. Unlike DG44 cells which had initially been cultured in 450 mOsm/kg media, cells placed directly in media with an osmolality of 550 mOsm/kg or greater showed poor growth and viability.

### Example 6

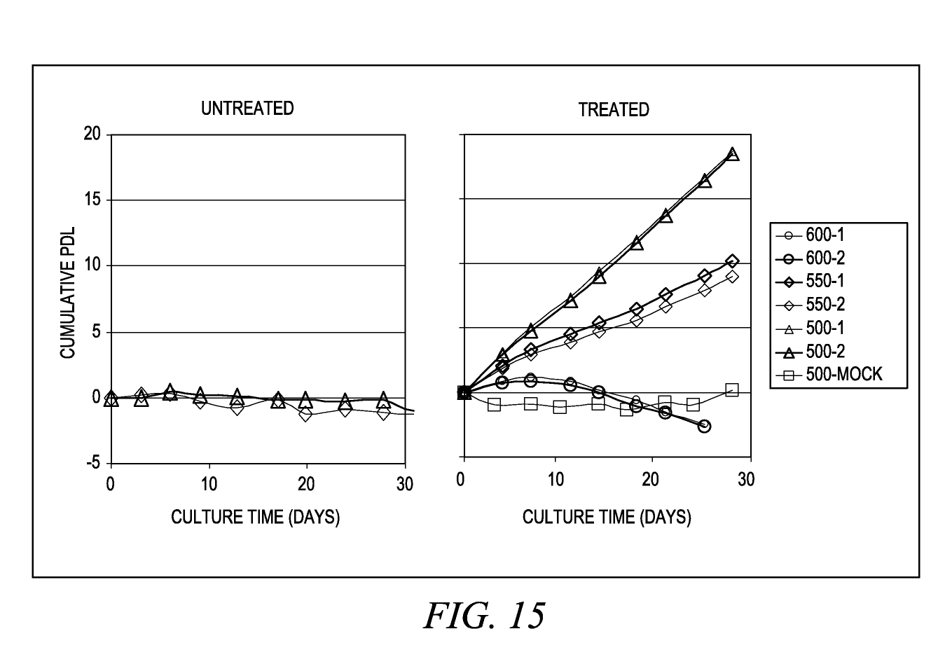

The pEF1-V5-HisA plasmid (Invitrogen, Carlsbad Calif., catalog no. V92020) may be digested by ScaI and transfected according to the Invitrogen manual into parental DG44 cells and DG44 cells in which the MMR system has been disrupted as described in U.S. Pat. Nos. 6,825,038 and 6,576,468. Two days later, 500 μg/ml G418 may be added to the medium for selection. Twenty-five days later, cell viability may have recovered to above 95%, and two weeks later, the cells may be transferred straight into medium adjusted to 500, 550 or 600 mOsm/Kg. Cells may be seeded at 0.3×10⁶/ml in 50 ml medium. A 1.5 ml sample may be taken every day to check cell density and viability. Exemplary results from such an experiment are shown in FIG. 15.

### Example 7

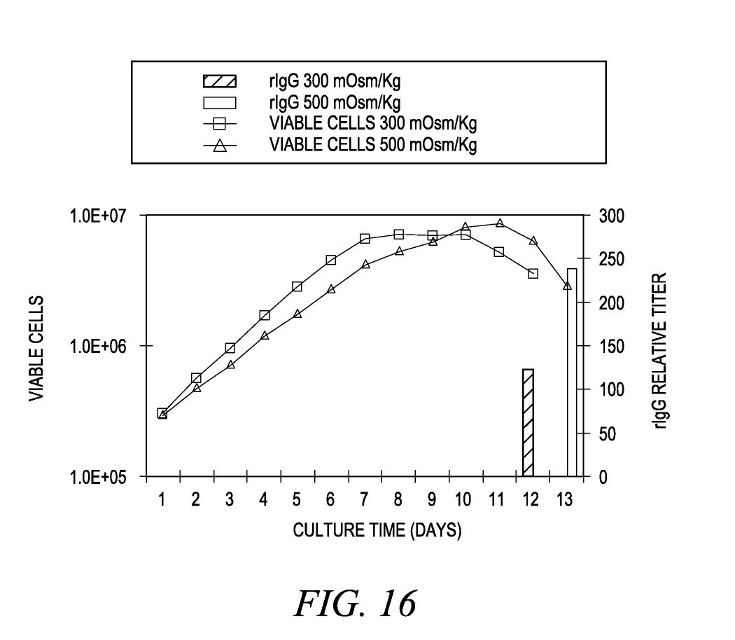

DG44 cells in which the MMR system had been disrupted as described in U.S. Pat. Nos. 6,825,038 and 6,576,468 and selected for tolerance to 500 mOsm/kg and able to cycle between 300 and 500 mOsm/kg were subsequently transfected with a plasmid containing the IgG gene. After selection and cell viability had recovered to above 95%, a portion of the recovered cells were switched to 500 mOsm/Kg in CD OptiCHO medium (Invitrogen Corp., Carlsbad, Calif.). After the cells had recovered from the shift, the IgG expression levels at both 300 and 500 mOsm/Kg conditions were tested. Cells were seeded at 0.3×10⁶/ml in 50 ml medium, a 1.5 ml sample was taken every day to check cell density and viability, the remaining sample was centrifuged and supernatant was frozen at −20° C. until ELISA assay for IgG concentration was performed. The results of this experiment are shown in FIG. 16.

Although the invention has been described with reference to the above example, it will be understood that modifications and variations are encompassed within the spirit and scope of the invention. Accordingly, the invention is limited only by the following claims.

What is claimed is:

1. A method for improving the growth properties of cells in culture comprising:
a) culturing cells under conditions wherein the mutation rate is increased;
b) adjusting the culture conditions such that a parameter is not optimal for the cell;
c) decreasing the mutation rate of the cells; and
d) isolating cells which maintain growth and viability under the adjusted culture conditions.

2. The method of claim 1, wherein the mutation rate of the cells is decreased prior to adjusting the culture conditions.

3. The method of claim 1, wherein the mutation rate is increased by inhibition or inactivation of the mismatch repair system.

4. The method of claim 1, wherein the adjusted culture condition parameter is selected from the group consisting of low osmolality, high osmolality, low pH, high pH, lack of essential nutrients or growth factors, accumulation of metabolic byproducts, high temperature, low temperature, presence of a solid support for cell attachment, lack of a solid support for cell attachment, exposure to high shear forces, low oxygen tension, and high carbon dioxide concentration.

5. The method of claim 1, wherein the mutation rate is increased for from 10 population doublings to 60 population doublings.

6. The method of claim 5, wherein the mutation rate is increased for from 10 population doublings to 50 population doublings.

7. The method of claim 6, wherein the mutation rate is increased for from 10 population doublings to 30 population doublings.

8. The method of claim 1, wherein the adjusted culture conditions are maintained for from 10 population doublings to 60 population doublings.

9. The method of claim 8, wherein the adjusted culture conditions are maintained for from 10 population doublings to 50 population doublings.

10. The method of claim 9, wherein the adjusted culture conditions are maintained for from 10 population doublings to 30 population doublings.

11. The method of claim 4, wherein the adjusted culture condition parameter is high osmolality.

12. The method of claim 11, wherein high osmolality is from 350 mOsm/kg to 800 mOsm/kg.

13. The method of claim 12, wherein high osmolality is from 350 mOsm/kg to 600 mOsm/kg.

14. The method of claim 12, wherein high osmolality is from 450 mOsm/kg to 800 mOsm/kg.
